(19) United States
(12) Patent Application Publication
(10) Pub. No.: US 2010/0124540 A1
(43) Pub. Date: May 20, 2010

(54) SKIN COMPOSITIONS

(76) Inventors: Gordon Gerald Guay, Chelmsford, MA (US); Ronald Patrick McLaughlin, Reading, MA (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/477,271

(22) Filed: Jun. 3, 2009

Related U.S. Application Data
(60) Provisional application No. 61/115,741, filed on Nov. 18, 2008.

(51) Int. Cl. A61K 8/81; A61Q 5/06

(52) U.S. Cl. 424/70.16

(57) ABSTRACT

A skin composition for use with or without a device such as a razor or a light based device (e.g., a laser) is provided in the present invention. The composition when exposed to light having a red wavelength is capable of substantially transmitting or not substantially absorbing or reflecting the red wavelength of light to protect the skin. Ingredients in the composition may be segmented into functional groups: a drying group, a skin benefits group, a performance group and a carrier group where each group has respective functional agents or ingredients. A container encompassing the composition may be integrated with a razor or light based device for ease of dispensing. The composition is quick-drying. After evaporation, there may be one or more non-volatile components of the composition remaining on the skin providing skin benefits.

### CROSS REFERENCE TO RELATED APPLICATION

This application claims the benefit of U.S. Provisional Application No. 61/115,741, filed Nov. 18, 2008.

### FIELD OF THE INVENTION

The field of the invention is liquid compositions (or formulations) and more particularly, liquid compositions for use on skin and that may also be capable of being used with any razor or light based devices, such as a laser device for hair growth management.

### BACKGROUND OF THE INVENTION

Liquid compositions for topical use are generally known in the art. The majority of the current liquid compositions have mineral oil and/or poly-glycol based materials. Oil and glycolic solutions may contain non-volatile ingredients and as such, generally need to be removed after use because they are typically oily and sticky to the touch.

Prior art liquid compositions or lotions range in the composition space or spectrum from wet to dry, and from oily to greasy. For instance, a wet lotion such as the Purell™ Hand Sanitizer product is on the opposite side of the composition or formulation spectrum from a product such as Coppertone Dry Sun which is typically considered a drier lotion. Oily lotions include moisturizers and baby oils, baby oil mists or gels. Greasy lotions in the composition space include petrolatum and viscous mineral oil products.

Light based products or light mediated devices or systems generally utilize low level optical radiation and are commonly known as laser devices. Many physician's offices, spas and salons use these laser devices for removal or management of unwanted hair as an alternative to waxing or electrolysis. Currently, there are liquid compositions marketed and intended for use with such light based products. These liquid compositions generally may assist in the functionality of the light based product and/or offer an aesthetic benefit to the consumer receiving the light based treatment in a professional services setting (e.g., in a physician's office, spa, or salon). Many times these liquid compositions are intended to be applied in direct preparation for or during a session with such a light based device.

One prior art liquid composition is the Palomar LuxLotion™ which is a lotion that may be considered oily or sticky and is currently being marketed and intended for use with a laser hair removal device. It has properties such that when applied to the skin provides optical coupling of light by evenly delivering light to the skin. Although the LuxLotion™ may meet many desired engineering requirements associated with concurrent use with a light based hair management device, it does not provide many aesthetic benefits essential for a viable consumer product, in that after applying and using the LuxLotion™ there is a need for the user to wipe or wash the lotion off the skin, which creates an untidiness after lotion application. Further, the lotion is generally not easy to dispense since it is typically dispensed by hand.

Thus, while many current liquid compositions may function properly with a laser device, the ingredients used in the formulation do not generally fulfill all of a user's desired requirements for use with a light based product. In many instances, the disadvantage of the prior art liquid compositions is that they do not provide aesthetic benefits desirable by consumers. There are three basic but desired aesthetic benefits for a user with regard to liquid compositions (or lotions) after application onto the skin. One desired benefit is that, after application of the lotion, there be no washing or wiping off necessary to remove the lotion from their skin. This saves a step for the user, thereby allowing the user to subsequently quickly use a razor or a light based device, for instance, to remove unwanted hair.

A second desired aesthetic benefit that follows from the first benefit mentioned above is that there be no mess after use of the lotion. “No mess” generally signifies that users do not have to touch the composition (i.e., hands free) in order to apply it and that the composition itself will not stain surfaces it comes into contact with, such as clothes, carpets, or linens. Accordingly, it is desired that the lotion be volatile or have volatile components.

A third desired benefit is that a user has an easy way to dispense the liquid composition onto their skin. For instance, the container holding the lotion could be integrated with the light based device and/or the lotion flows easily from the container, not requiring the user to expend a lot of energy to dispense lotion.

Thus, a liquid composition is desired that provides the three aesthetic benefits mentioned above in conjunction with the use of a light mediated device, such as, but not limited to, a hair growth management system. In addition, however, to making the system or device appealing to users, there are a number of engineering criteria for the composition that are desired in order to be used optimally with a light mediated hair management system or other light based device.

### SUMMARY OF THE INVENTION

The invention provides a skin composition including one or more ingredients wherein, when the skin composition is exposed to light having a red wavelength, the composition substantially transmits, or does not substantially absorb or reflect the red wavelength of light.

In one embodiment, the ingredients include water at about 55% by weight of composition, a silicone emulsion at about 25% by weight of composition, ethanol at about 10% by weight of composition, glycerin at about 2% by weight of composition, passiglora edulis seed oil at about 2% by weight of composition, butylene glycol at about 2% by weight of composition, glycereth-18 ethylhexanoate at about 1.5% by weight of composition, glycerin glyceryl acetate/acrylic acid copolymer, polyvinyl methyl/maleic anhydride copolymer (lubrajel oil) at about 1% by weight of composition, and phenoxyethanol at about 0.5% by weight of composition and fragrance at about 0.4% by weight of composition.

In another embodiment, the ingredients include water from about 40% to about 60% by weight of composition, a silicone emulsion from about 10% to about 45% by weight of said composition, about 20% by weight of said composition of ethanol, up to about 20% by weight of composition of glycerin, up to about 20% by weight of composition of hydramazon passiflora, and up to about 20% by weight of composition of phytexcell jujube, up to about 5% by weight of composition of glycerin glyceryl acetate/acrylic acid copolymer, PVM/MA copolymer (lubrajel oil) and up to about 5% by weight of composition of glycereth-18 ethylhexanoate.

In one aspect, the skin composition is integrated in a light based device and may include a sensory cue. In another aspect, the skin composition is quick-drying. In yet another aspect, one or more non-volatile components of the composition remain on a user's skin after evaporation, the non-volatile components remaining ranging from about 0.01% to about 20% by weight of composition.

The skin composition includes a drying agent, a skin benefit agent, a performance agent, and a carrier agent, wherein when the composition is exposed to light having a red wavelength, the composition substantially transmits, or does not substantially absorb or reflect the red wavelength of light. The drying agent includes a silicone emulsion and an ethanol. The skin benefit agent is selected from the group consisting of glycerin, hydramazon passiflora, phytexcell jujube, and combinations thereof. The performance agent is selected from the group consisting of glycerin glyceryl acetate/acrylic acid copolymer, PVM/MA copolymer (lubrajel oil), glycereth-18 ethylhexanoate, and combinations thereof. The carrier agent includes water.

In the present invention, the silicone emulsion is from about 10% to about 45% by weight of the skin composition, the ethanol is at about 20% by weight of the composition, the glycerin is up to about 20% by weight of composition, the hydramazon passiflora is up to about 20% by weight of composition, and the phytexcell jujube is up to about 20% by weight of composition, the lubrajel oil is up to about 5% by weight of composition and said glycereth-18 ethylhexanoate is up to about 5% by weight of composition, and the water is from about 40% to about 60% by weight of composition.

In another aspect of the invention, the skin composition includes a plurality of ingredients wherein one or more non-volatile components of the composition remain on a user's skin after evaporation, the non-volatile components remaining ranging from about 0.01% to about 20% by weight of composition. The solids remaining is about 3.5% by weight of composition. The composition is quick-drying. When the skin composition is exposed to light having a red wavelength, the composition substantially transmits, or does not substantially absorb or reflect the red wavelength of light.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

The novel liquid composition provides aesthetic benefits to the user, in that there is no washing or wiping off necessary to remove the lotion from their skin, there is no mess (i.e., “hands-free”) and there is a simple way to dispense the composition through a container or a device.

The novel liquid composition may also provide other functional skin, hair or laser use benefits such as being medically safe, providing relief from discomfort, cooling the skin, minimizing hair on the skin, and toning the skin.

The formulary space of the present invention represents a liquid composition that may, if necessary, be used with a light based device such as with a laser based hair management system.

For use with a light based device, the composition of the present invention shall additionally be optically transparent and does not substantially reflect light in the red wavelength of light which is at or about 600-1000 nm. The red wavelength of light becomes significant in conjunction with the use of light based devices since the liquid composition will absorb light energy (or optical radiation) from the light based device and hence generate heat at the user's skin surface. This heat could result in skin surface “burns” or red marks, which are not desired as they may cause injury and moreover, may also block or lessen the amount of light reaching the desired location to produce the ideal effect, e.g., hair follicle location and penetration in the skin in the case of the hair growth management laser device.

Additionally, the liquid composition shall also desirably provide a nice glide and tracking for users of such devices, while also not negatively interfering with the device's safety or velocity sensors (e.g., such as those associated with motion and contact) or other device mechanisms.

In addition to the above-mentioned desired aesthetic benefits appealing to users during and after application, the composition of the present invention has a low viscosity and be properly dispensable in low, tailorable or adequate quantities to the skin during use.

The composition of the present invention may be delivered to the skin in any manner. One mechanism may include a device (e.g., a razor or a light based device) having an integrated lotion and a delivery mechanism to dispense and/or tailor the amount of composition being dispensed. Alternatively, the composition may be delivered to the skin from a container which has its own dispensing (e.g., a pump).

Thus, the composition space defined (e.g., hydro alcoholic) of the present invention represents an area of chemistry that fulfills all of the engineering and aesthetic requirements outlined within the objectives above.

It should be noted that the terms “formulation” and “composition” are intended to have an equivalent meaning and will be used interchangeably herein. Additionally, the terms “liquid”, “lotion” or “topical” will also be used herein interchangeably as having equivalent meanings. Also, “hair management”, “hair removal”, and “hair growth management” may be used interchangeably and herein are deemed equivalents.

In an embodiment of the present invention, a novel liquid composition capable of being used with a razor or a light based device or system combines ingredients as shown in the Examples. As will be described below, this new formulation may have a hydro-alcoholic volatile silicone blend and uniquely delivers a composition that provides many beneficial attributes.

The compositions of the present invention provide the key to delivering the benefits desired, such as good lubrication, lack or reduced irritation, and no mess or nothing left on the skin for the user to wipe off. Additionally, a functional benefit when used concurrently with a laser device on the skin which may have a red wavelength of light (at or about 600 to l000 nm) is that the composition does not absorb or substantially reflect the red wavelength of light.

The ingredients in the novel liquid composition may be grouped into, though not limited to, four categories according to their functionality: (1) a drying group; (2) a skin care or skin benefit group; (3) a performance group; and (4) a carrier group. The groups have agents or ingredients that are dermatologically-acceptable and the composition of the present invention, regardless of groupings or combinations, is capable, when exposed to light having a red wavelength, of substantially reflecting the red wavelength (at or about 600 to 1000 nm) of light.

Ingredients or agents in the drying group allow for controlled evaporation or drying of the lotion on the skin. Ingredients in the skin benefits group provide the moisturizing and soothing benefits found with glycerin, emollients, humectants, and polymers. Ingredients or agents in the performance group generally provide lubricity or better moisturizing results. The carrier group agents or ingredients generally represent the formulary chassis, typically an aqueous base as noted in the Examples.

The drying group of the composition may utilize drying agents such as a volatile silicone or silicone emulsion such as cyclomethicone (DC245) ingredient to provide glide, positive after-feel and desirably a controlled evaporation or drying time, while alcohol reduces tack (e.g., stickiness) and cools the skin. The DC245 agent may include about 5-15% PEG-12, about 20-30% cyclopentasiloxane, about 0.1-2% sodium lauryl ether sulfate (SLES), and about 60-80% water. An exemplary formulation may include DC245 at or about 25% by weight of the composition of the present invention.

Another drying agent of the composition of the present invention may include alcohol. The alcohol concentration provides a sensory cue (e.g., evaporative cooling), enabling a user to feel the location where the razor or light based product (or laser) was applied on their skin. A type of menthol may also be used to provide this cooling sensation. A suitable formulation of the present invention may have ethanol at or about 10% by weight of the composition. The alcohol agent also affects the ability of the composition to dry by evaporating or drying on a user's skin quickly, such that a user does not have to wipe up the composition.

The skin benefits or skin care group of the composition includes agents such as emollients, humectants and polymers that are formulated into the composition to deliver skin, shaving, and light based benefits. The skin benefits group provides moisturization and may include agents as indicated in the Examples, glycerin and/or other extracts passiglora edulis seed oil—polyethylene glycol (PEG) 8 esters and passiflora incarnata seed oil—PEG 8 esters (hydramazon passiflora) and butylene glycol, and zizphus jujuba fruit extract (phytexcell jujube).

The performance group of the composition of the present invention includes performance agents providing lubricity or moisture, such as glycereth-18 ethylhexanoate and glycerin and glyceryl acrylate/acrylic acid copolymer and propylene glycol and polyvinyl methyl/maleic anhydride (PVM/MA) copolymer (i.e., lubrajel oil).

The carrier group of the composition of the present invention includes any dermatologically-acceptable carrier agent such as water which provides an aqueous base.

It should be noted that the ingredients or agents in the present invention composition may fall into more than one of the functional groups mentioned above, as they may provide dual or overlapping benefits.

After application of the formulation onto a user's skin, the dispensed lotion is visible and perceptible for a finite period of time and then dries so there is no washing or wiping off necessary to remove the composition from a user's skin. The present invention composition is not oily or sticky. This characteristic saves a step for the user, allowing the user to quickly finish using the razor or light based product to remove unwanted hair.

Furthermore, there is no mess after use of the lotion composition. “No mess” as mentioned above, generally signifies that users do not have to touch the composition (i.e., “hands free”) in order to apply it and that the composition itself will not stain surfaces it comes into contact with, such as clothes, carpets, or linens. The composition of the present invention can be dispensed through direct application on the skin by the user's hand (if desired), by use with a light based device or system, or by another hand held applicator (e.g., pump container). For instance, the container holding the lotion could be integrated with the light based device such that the lotion flows easily from the container, not requiring the user to expend a lot of energy to dispense.

This liquid composition provides a nice glide, while also not negatively interfering with the device's safety sensors associated with motion and contact. Additionally, the novel composition represents an area of formulary space (i.e. fragrance, color, texture) that is appealing to users during and after application with low viscosity and likewise capable of being properly dispensed in low quantities to the skin during use.

Additionally, in accordance with a desirable aspect of the present invention, the liquid composition of the present invention provides product tracking such that it is visible to the user particularly with the concurrent use of a laser device (e.g., for the user to track or see and know which skin areas have been covered and where a laser based system has been applied). This essentially acts as a visible tracking signal (e.g., a sensory cue) that should fade out without requiring user action. The visibility in the present invention may last for from about a few seconds to two to three minutes or less after application with a laser device. It effectively disappears. As such, the aesthetic benefit of tracking is provided while also providing the advantage of having a reasonably quick lotion drying time, thereby providing another desired aesthetic benefit since a user does not have to use their hands or other means to wipe or wash off the lotion once it has already dried.

In an alternate embodiment of the present invention, formulation properties may be altered without loss of any of the aforementioned benefits, such as no mess, nice glide, visible, volatile, etc.

One instance that may necessitate alterations to the concentrations and ingredients of the present formulation depends on the manner in which the lotion is delivered to the skin. For example, if the lotion is not dispensed through a device, the volatility of the lotion may be modified to improve slippage. On the contrary, if the lotion is dispensed through a delivery mechanism integrated with the device, the viscosity of the lotion may be modified taking into account the interaction with the delivery mechanism and the applicator surface.

Any purpose may warrant a modification of the concentration levels in the formulation. Modifications encompassing the contemplated ranges of concentrations of the compositions of the present invention are indicated in the Examples. Other concentrations are also contemplated in the present invention which may provide similar benefits to the user with or without concurrent use of a device (e.g., a razor or a laser device).

Therefore, the composition of the present invention can also be used in conjunction with a razor or a light based device and may be dispensed through a separate container or direct application. Likewise, compositions of the present invention may also be modified as desired; for instance, to include permethyls or silicone or other agents.

In another aspect of the present invention, the composition is desirably quick-drying. As used herein, quick-drying signifies a composition which has substantially evaporated in a short amount of time. Thus, in another aspect of the invention, the compositions within the scope of the present invention may include a quick-drying aspect including a volatility profile whereby after about one minute, at least half or 50% of the liquid will have dried or evaporated. After about 5 minutes or desirably after about 1-2 minutes or within the range of 10 seconds to 10 minutes, the evaporation of the composition of the present invention may be entirely complete with only non-volatile components remaining.

The percentage of non-volatile components remaining in the present invention is at or about 0.01% to 20% by weight of composition and desirably about 3.5% for providing post-use benefits to the consumer. The types of solids remaining may represent skin conditioners, bio-actives, shine/gloss enhancers, hair-minimizers, moisturizers or other ingredients desirably left behind by the composition on the skin following skin composition (lotion) application.

### EXAMPLES

It should be noted that the Examples below illustrate embodiments described above within the scope of the present invention and are given solely for the purpose of illustration and are not to be construed as limitations of the present invention as many variations of the invention are possible without departing from its spirit and scope.

Unless otherwise indicated, all ingredients are expressed as a weight percentage of the active ingredient.

### Example 1

A composition of the present invention is prepared by combining the following ingredients.

| Ingredient | % w/w | Function |
|---|---|---|
| water, deionized | 55.600 | medium |
| D5 silicone emulsion | 25.000 | slip, after feel |
| (5-15% polyethylene glycol (PEG)-12, | | |
| 20-30% cyclopentasiloxane, 0.1-2% SLES, | | |
| 60-80% water) | | |
| ethanol | 10.000 | cooling, reduce |
| | | tack |
| glycerin | 2.000 | humectant |
| passiglora edulis seed oil; | | anti-irritant |
| polyethylene glycol (PEG) 8 esters; | | |
| passiflora incarnata seed oil; | 2.000 | |
| polyethylene glycol PEG 8 esters | | |
| (hydramazon passiflora) | | |
| butylene glycol, zizphus jujuba fruit extract | 2.000 | anti-irritant |
| (phytexcell jujube) | | |
| glycereth-18 ethylhexanoate | 1.500 | emollient |
| glycerin; | | slip, moisturizing |
| glyceryl acrylate/acrylic acid copolymer; | | |
| propylene glycol and polyvinyl methyl/ | 1.000 | |
| maleic | | |
| anhydride, polyvinyl methyl/maleic | | |
| anhydride (PVM/MA) copolymer | | |
| (lubrajel oil) | | |
| phenoxyethanol | 0.500 | preservative |
| fragrance (e.g., meadow medley) | 0.400 | odor, scent |

### Example 2

A composition of the present invention is prepared by combining the following ingredients.

| Ingredient | % w/w | Function |
|---|---|---|
| water, deionized | 40.0-60.0 | medium, base |
| cyclomethicone D5 silicone emulsion | 10.0-45.0 | slip, after-feel, |
| | | dry |
| ethanol | 0.0-20.0 | cooling, reduce |
| | | tack/stickiness |
| glycerin (or any other water-soluble | 0.0-20.0 | humectant, skin |
| humectant) | | |
| hydramazon passiflorae | 0.0-20.0 | anti-irritant, skin |
| (or any other water solubl extract) | | |
| phytexcell jujube, croda (or any other | 0.0-20.0 | anti-irritant, skin |
| water soluble extract) | | |
| glycereth-18 ethylhexanoate (or any | 0.0-5.0 | emollient, |
| other water soluble emollient) | | performance |
| glycerin; | 0.0-5.0 | slip, moisturizing |
| glyceryl acrylate/acrylic acid | | |
| copolymer; propylene glycol; and | | |
| polyvinyl methyl/ maleic anhydride | | |
| (PVM/MA) copolymer (or any other | | |
| water soluble polymer) | | |
| phenoxyethanol | 0.500-1.0 | preservative |
| any fragrance (any fragrance) | 0.0-1.0 | odor, pleasant |
| | | scent |
| | 100.000 | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A skin composition comprising one or more ingredients wherein, when said composition is exposed to light having a red wavelength, said composition substantially transmits, or does not substantially absorb or reflect said red wavelength of light.

2. The composition of claim 1 wherein said ingredients further comprise water at about 55% by weight of composition, a silicone emulsion at about 25% by weight of composition, ethanol at about 10% by weight of composition, glycerin at about 2% by weight of composition, passiflora edulis seed oil at about 2% by weight of composition, butylene glycol at about 2% by weight of composition, glycereth-18 ethylhexanoate at about 1.5% by weight of composition, glycerin glyceryl acetate/acrylic acid copolymer, polyvinyl methyl/maleic anhydride copolymer (lubrajel oil) at about 1% by weight of composition, and phenoxyethanol at about 0.5% by weight of composition and fragrance at about 0.4% by weight of composition.

3. The composition of claim 1 wherein said ingredients comprise water from about 40% to about 60% by weight of composition, a silicone emulsion from about 10% to about 45% by weight of said composition, about 20% by weight of said composition of ethanol, up to about 20% by weight of composition of glycerin, up to about 20% by weight of composition of hydramazon passiflora, and up to about 20% by weight of composition of phytexcell jujube, up to about 5% by weight of composition of glycerin glyceryl acetate/acrylic acid copolymer, PVM/MA copolymer (lubrajel oil) and up to about 5% by weight of composition of glycereth-18 ethylhexanoate.

4. The composition of claim 1 wherein said skin composition is integrated in a device.

5. The composition of claim 4 wherein said skin composition comprises a sensory cue.

6. The composition of claim 1 wherein said skin composition is quick-drying.

7. The composition of claim 1 wherein one or more non-volatile components of said composition remain on a user's skin after evaporation, said non-volatile components remaining ranging from about 0.01% to about 20% by weight of composition.

8. A skin composition comprising:
a drying agent;
a skin benefit agent;
a performance agent; and
a carrier agent, wherein when said composition is exposed to light having a red wavelength, said composition substantially transmits, or does not substantially absorb or reflect said red wavelength of light.

9. The composition of claim 8 wherein said drying agent comprises a silicone emulsion.

10. The composition of claim 8 wherein said drying agent comprises an ethanol.

11. The composition of claim 8 wherein said skin benefit agent is selected from the group consisting of glycerin, hydramazon passiflora, phytexcell jujube, and combinations thereof.

12. The composition of claim 8 wherein said performance agent is selected from the group consisting of glycerin glyceryl acetate/acrylic acid copolymer, PVM/MA copolymer (lubrajel oil), glycereth-18 ethylhexanoate, and combinations thereof.

13. The composition of claim 8 wherein said carrier agent comprises water.

14. The composition of claim 9 wherein said silicone emulsion is from about 10% to about 45% by weight of said composition.

15. The composition of claim 10 wherein said ethanol is at about 20% by weight of said composition.

16. The composition of claim 11 wherein said glycerin is up to about 20% by weight of composition, said hydramazon passiflora is up to about 20% by weight of composition, and said phytexcell jujube is up to about 20% by weight of composition.

17. The composition of claim 12 wherein said lubrajel oil is up to about 5% by weight of composition and said glycereth-18 ethylhexanoate is up to about 5% by weight of composition.

18. The composition of claim 13 wherein said water is from about 40% to about 60% by weight of composition.

19. A skin composition comprising:
a plurality of ingredients wherein one or more non-volatile components of said composition remain on a user's skin after evaporation, said non-volatile components remaining ranging from about 0.01% to about 20% by weight of composition.

20. The composition of claim 19 wherein said non-volatile components remaining is about 3.5% by weight of composition.

21. The composition of claim 19 wherein said composition is quick-drying.

22. The composition of claim 19 wherein said non-volatile components remaining provide skin benefits to the user.

23. The composition of claim 19 wherein when said composition is exposed to light having a red wavelength, said composition substantially transmits, or does not substantially absorb or reflect said red wavelength of light.
